(19) United States
(12) Patent Application Publication
(10) Pub. No.: US 2006/0280314 A1
(43) Pub. Date: Dec. 14, 2006

(54) Digital amplifier and switching power supply

(75) Inventors: Teruo Okada, Gunma (JP); Wataru Katada, Gunma (JP); Tatsuya Sakurai, Gunma (JP)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(73) Assignee: TAIYO YUDEN CO., LTD.

(21) Appl. No.: 11/376,805

(22) Filed: Mar. 15, 2006

(30) Foreign Application Priority Data
Jan. 10, 2006 (JP) 002531/2006
Mar. 16, 2005 (JP) 075421/2005

(51) Int. Cl. A61F 11/06; H04B 15/00

(52) U.S. Cl. 381/71.1; 381/94.1

(57) ABSTRACT

To provide a digital amplifier and a switching power supply which are effective to realize a noise level required for a high grade and output digital amplifier and a switching power supply. In an embodiment, a noise component extraction circuit is coupled to the output stage of a D-class driver. The noise component extraction circuit extracts a noise component included in the output of the D-class driver and adjusts the phase and gain of the extracted noise component. After its phase and gain have been adjusted, the extracted noise component is added to the output of a low-pass filter. As a result, the noise component remaining in an audio signal that has passed through the low-pass filter is canceled.

### CROSS REFERENCE TO RELATED APPLICATIONS

This application claims priority to Japanese Application No. 002531/2006, filed Jan. 10, 2006, which claims priority to Japanese Application No. 075421/2005, filed Mar. 16, 2005.

### FIELD OF THE INVENTION

The field of the invention relates to a digital amplifier and a switching power supply, and particularly to a digital amplifier and a switching power supply which are effective for noise reduction.

### DESCRIPTION OF THE RELATED ART

Recently, in the advanced multi-function/high-integration of audio-video (AV) systems, digitalization of a composite AV system has received attention. In this kind of composite AV system, an AV component in which a CD player, an AM/FM radio tuner, and an audio amplifier are formed in the same housing has been known.

Further, recently, as an audio amplifier incorporated in the above composite AV system, the change from an analog amplifier to a digital amplifier has been investigated. Since the use of the digital amplifier may result in size-reduction, low heat generation, and high-quality audio compared to the conventional analog amplifier, the digital amplifier is becoming the mainstream amplifier of the composite AV system.

Since amplification of an audio signal in this kind of digital amplifier is performed by a switching operation, a noise or distortion measure is important. Simultaneously, since a switching power supply also uses a switching power drive circuit similar to the digital amplifier, a residual component of a fundamental wave, which is referred to as carrier ripple noise, exists in the output stage of this switching circuit.

Since this residual ripple noise frequently constitutes unnecessary noise that has a bad influence on other circuits, reduction of the ripple noise is required. To reduce the ripple noise, a method of increasing the time constant or the second degree of an output-stage filter is generally applied.

However, increasing the time constant or the second degree of the filter adversely affects the cost and size of the filter. Therefore, it is desirable that the filter is formed with as minimum a time constant and second degree as possible.

Other example approaches to the noise reduction are disclosed in JP-A-2003-258565 and JP-A-2004-128662.

In JP-A-2003-258565, the following constitution and method are indicated: when a CD player plays a CD, a digital amplifier is driven; when the radio is received, the digital amplifier is stopped and an analog amplifier is used; and the digital amplifier is put in a shielding case to be shielded.

Further, In JP-A-2004-128662, the following method is disclosed: an audio input signal and an output signal in an output amplification stage are compared, and the output of a constant voltage power circuit is modulated on the basis of this comparison result, whereby the distortion in the output amplification stage can be reduced.

It is thought that both of the methods disclosed in the above related arts are effective. However, in order to achieve a noise level necessary for a digital amplifier used for the purpose of high output and high grade, more noise reduction is desirable.

On the other hand, a method of canceling a pulse noise included in a high frequency signal is described in JP-A-2000-91932, JP-A-2000-91933, and JP-A-2000-101459.

In these related fields, a method is disclosed in which the signal down-converted into an intermediate frequency signal is taken out before detection, and a harmonic wave component is extracted and added to the down-converted signal after the detection, whereby the noise component is cancelled.

However, in the case that the noise cancellation method disclosed in these relates arts is applied to the digital amplifier and the switching power supply as it is, sufficient noise cancellation effect cannot be obtained. Therefore, in order to achieve the noise level required for the digital amplifier used for the purpose of high output and high grade, more improvement is necessary.

### BRIEF SUMMARY OF THE INVENTION

An advantage of an aspect of the invention is to provide a digital amplifier and a switching power supply which are effective to realize a noise level required for a digital amplifier and a switching power supply that are used for the purpose of high output and high grade.

According to a first separate aspect of the invention, a digital amplifier which switch-amplifies an audio signal and performs analog output, includes a pulse modulation circuit which modulates the audio signal; a D-class drive circuit which is driven by the output of the modulation circuit; a filter in which the output of the D-class drive circuit is smoothed; and a noise component extraction circuit which extracts a noise component from the output of the D-class drive circuit and adds the extracted noise component to the output of the filter. The noise component extraction circuit includes a unit for attenuating the extracted noise component correspondingly to an attenuation characteristic of the filter.

Thus, the noise component is attenuated correspondingly to the attenuation characteristic of the filter provided in the output stage of the D-class drive circuit, whereby the level of each of the various kinds of noise components interspersed in an attenuation area of the filter can be reproduced in the noise component extraction circuit. Therefore, the noise reducing accuracy can be improved.

Particularly, in the digital amplifier, a switching frequency in the D-class drive circuit varies with a band width. Therefore, the switching noises in the D-class drive circuit after passing through the filter are different in level in the band, and they cannot be removed sufficiently by the noise cancellation method that has been conventionally known.

The extracted noise component, in order to obtain a noise cancellation effect, is added to an audio signal after the phase of the extracted noise component has been inverted. Namely, exemplifying a case where a ripple noise caused by the switching operation is cancelled, the noise component extraction circuit firstly extracts a switching carrier component, inverts the phase of this extracted carrier component, matches the amplitude level of the carrier output with the amplitude level of the filter path output, and adds the carrier component to the audio signal that has been output from the filter.

Further, according to a second separate aspect of the invention, a digital amplifier which switch-amplifies an audio signal and performs analog output, includes a pulse modulation circuit which modulates the audio signal; a D-class drive circuit which is driven by the output of the modulation circuit; a filter that smooths the output of the D-class drive circuit; and

a noise component extraction circuit which extracts a noise component from the output of the D-class drive circuit and adds the extracted noise component to the output of the filter. Herein the output of the noise component extraction circuit is added through a DC cut capacitor to the output of the filter.

Thus, by adding the extracted noise component through the DC cut capacitor to the output of the filter, it is possible to suppress the drop in efficiency caused by addition of the noise cancellation function. Namely, in the case where there is no DC cut capacitor, the DC current flows due to offset and electric power is consumed, while in the case where there is the DC cut capacitor, the power consumption due to the DC current can be avoided.

In the case having the DC cut capacitor, the output level of the noise component extraction circuit lowers according to a partial pressure ratio to the capacitor constituting the filter. Therefore, it is preferable that an amplifier for supplementing the output level is provided. For example, in the case that the value of the DC cut capacitor is the same as that of the capacitor constituting the filter, the partial pressure ratio becomes one over two. Therefore, in the noise component extraction circuit or in the back stage of the circuit, an amplifier having double amplification factor is provided.

Further, according to a third separate aspect of the invention, a digital amplifier, which switch-amplifies an audio signal and has an analog output, includes a pulse modulation circuit which modulates the audio signal; a D-class drive circuit which is driven by the output of the modulation circuit; a filter of low-impedance output in which the output of the D-class drive circuit is smoothed; and a noise component extraction circuit which extracts a noise component from the output of the D-class drive circuit and adds the extracted noise component to the output of the filter. The noise component extraction circuit includes a phase compensation circuit which compensates a phase error produced by addition of the extracted noise component to the low impedance output of the filter.

Thus, by providing the phase compensation circuit, the noise components are cancelled in a phase-matching state even in the case that the output of the noise component extraction circuit is added to the low impedance line. Therefore, it is possible to obtain the noise cancellation effect which is effective also for the digital amplifier having the low impedance output.

Further, according to a fourth separate aspect of the invention, a digital amplifier, which switch-amplifies an audio signal and has an analog output, includes a pulse modulation circuit which modulates the audio signal; a D-class drive circuit which is driven by the output of the modulation circuit; a filter in which the output of the D-class drive circuit is smoothed; and

a noise component extraction circuit which extracts a noise component from the output of the D-class drive circuit and adds the extracted noise component to the output of the filter. The noise component extraction circuit includes: a band limiting part which extracts the noise component; a phase compensation part which compensates the phase of the extracted noise component; and a gain adjustment part which adjusts the gain of the extracted noise component correspondingly to an attenuation characteristic of the filter.

Thus, by providing the band limiting part, the phase compensation part, and the gain adjustment part in the noise component extraction circuit, the noise component can be selectively extracted, and the level and the phase of the noise component remaining after the audio signal has passed through the filter can be matched with the level and the phase of the noise component extracted for the purpose of cancellation. Therefore, the effect of more accurate noise cancellation can be obtained.

Further, according to a fifth separate aspect of the invention, a digital amplifier, which switch-amplifies an audio signal and has an analog output, includes a pulse modulation circuit which modulates the audio signal; a D-class drive circuit which is driven by the output of the modulation circuit; a filter in which the output of the D-class drive circuit is smoothed; and

a noise component extraction circuit which extracts a noise component from the output of the D-class drive circuit and adds the extracted noise component to output of the filter. The noise component extraction circuit includes a high-pass filter which extracts the noise component, and a low-pass filter having the same attenuation characteristic as the attenuation characteristic of the aforesaid filter.

Thus, by constituting the noise component extraction circuit by means of the high-pass filter and the low-pass filter, band limiting, phase compensation, and gain adjustment which are important for noise cancellation can be performed preferably. Namely, by the high-pass filter, the noise component is extracted, and the phase of this extracted noise component is compensated; and by the attenuation characteristic of the low-pass filter, the level of the noise component is adjusted, so that the noise component remaining in the output of the filter provided in the back stage of the D-class drive circuit is canceled with high accuracy.

Further, according to a sixth separate aspect of the invention, the noise component extraction circuit in the fifth separate aspect further includes an amplifier which amplifies the output level of the low-pass filter, and a DC cut capacitor which cuts a DC component from the output of the low-pass filter.

Thus, by providing the amplifier for output-level adjustment and the capacitor for cutting the DC component, in a state where the noise level necessary for noise cancellation is kept, it is possible to prevent power consumption due to the DC current.

Further, according to a seventh separate aspect of the invention, a digital amplifier which switch-amplifies an audio signal and has an analog output, includes a pulse modulation circuit which modulates the audio signal; a D-class drive circuit which is driven by the output of the modulation circuit; a filter in which the output of the D-class drive circuit is smoothed; and

a noise component extraction circuit which extracts a noise component from the output of the D-class drive circuit thereby to generate a reverse-phase noise component, and adds this reverse-phase noise component to the output of the filter.

Thus, by extracting the noise component from the output of the D-class drive circuit and adding the noise component to the output of the filter, the unnecessary noise component included in the output of the D-class drive circuit can be extracted as a cancellation target and added to the audio signal in reverse phase. Therefore, the necessary noise such as the switching noise remaining in the audio signal due to the switching operation of the D-class drive circuit can be reduced.

Further, according to an eighth separate aspect of the invention, the noise component extraction circuit, in the seventh aspect, is decoupled to the output of the D-class drive circuit.

Thus, by decoupling a main path in which the audio signal is generated from a noise extraction path, the input of large current to the noise component extraction circuit can be avoided.

Further, according to a ninth separate aspect of the invention, a digital amplifier which switch-amplifies an audio signal and performs analog output, includes a pulse modulation circuit which modulates the audio signal; a D-class drive circuit which is driven by the output of the modulation circuit; a filter in which the output of the D-class drive circuit is smoothed; and

a noise component extraction circuit which extracts a noise component from the output of the filter thereby to generate a reverse-phase noise component, and adds this reverse-phase noise component to the output of the filter.

Thus, by extracting the noise component from the output of the filter and adding the noise component to the output of the filter, the unnecessary noise component included in the output of the filter can be extracted as a cancellation target and added to the audio signal in reverse phase. Therefore, the necessary noise such as the switching noise remaining in the audio signal due to the switching operation of the D-class drive circuit can be reduced.

Further, according to a tenth separate aspect of the invention, the noise component extraction circuit is decoupled from the output of the filter.

Thus, by decoupling a main path in which the audio signal is generated from a noise extraction path, the input of large current to the noise component extraction circuit can be avoided, and an influence exerted on the main path by the noise extraction path can be reduced.

Further, according to an eleventh separate aspect of the invention, a switching power supply which controls a switching element arranged between a power source and a load thereby to perform power conversion, includes a control circuit which controls the switching element; a filter in which the output of the switching element is smoothed; and a noise component extraction circuit which extracts a noise component from the output of the switching element thereby to generate a reverse-phase noise component, and adds this reverse-phase noise component to the output of the filter.

Thus, by extracting the noise component from the output of the switching element and adding the noise component to the output of the filter, the unnecessary noise component included in the output of the switching element can be extracted as a cancellation target and added to the power supply output in reverse phase. Therefore, the necessary noise such as the switching noise remaining in the power supply output due to the switching operation of the switching element can be reduced.

Further, according to a twelfth separate aspect of the invention, a switching power supply which controls a switching element arranged between a power source and a load thereby to perform power conversion, includes a control circuit which controls the switching element; a filter in which the output of the switching element is smoothed; and a noise component extraction circuit which extracts a noise component from the output of the filter thereby to generate a reverse-phase noise component, and adds this reverse-phase noise component to the output of the filter.

Thus, by extracting the noise component from the output of the filter and adding the noise component to the output of the filter, the unnecessary noise component included in the output of the filter can be extracted as a cancellation target and added to the power supply output in reverse phase. Therefore, the necessary noise such as the switching noise remaining in the power supply output due to the switching operation of the switching element can be reduced.

Further, according to a thirteenth separate aspect of the invention, a noise reduction circuit includes a switching unit for switching an input signal; a filter connected to the back stage of the switching unit; and a noise component extraction circuit, which extracts a noise component included in output of the switching unit from the output thereby to generate a reverse-phase noise component, and adds this reverse-phase noise component to the output of the filter.

Thus, by extracting the noise component from the output of the switching unit and adding the noise component to the output of the filter, the unnecessary noise component included in the output of the switching unit can be extracted as a cancellation target and added to the output of the filter in reverse phase. Therefore, the necessary noise such as the switching noise remaining in the filtered output due to the switching operation of the switching unit can be reduced.

Further, according to a fourteenth separate aspect of the invention, a noise reduction circuit includes a switching unit for switching an input signal; a filter connected to a back stage of the switching unit; and a noise component extraction circuit, which extracts a noise component included in output of the filter from the output thereby to generate a reverse-phase noise component, and adds this reverse-phase noise component to the output of the filter.

Thus, by extracting the noise component from the output of the filter and adding the noise component to the output of the filter, the unnecessary noise component included in the output of the filter can be extracted as a cancellation target and added to the output of the filter in reverse phase. Therefore, the necessary noise such as the switching noise remaining in the filtered output due to the switching operation of the switching unit can be reduced.

In the above-described constitution, the pulse modulation circuit may include a PWM translation circuit and a delta-sigma (ΔΣ) translation circuit. Further, the pulse modulation circuit may include a function of performing the predetermined calculation processing for the audio signal. As modulation used in the digital amplifier circuit, there are PWM (Pulse Width Modulation) and PDM (Pulse Density Modulation). As a data format inputted in the digital amplifier circuit, there is PCM (Pulse Code Modulation) used in music CDs.

The audio signal inputted in the pulse modulation circuit may be an analog signal or a digital signal. In the case that the analog signal is input, the analog signal is directly input to the pulse modulation circuit and converted into a pulse signal, or after the analog signal has been converted through an A/D converter into a digital signal once, it may be input to the pulse modulation circuit.

On the other hand, in the case that the digital signal is input, the digital signal is directly input to the pulse modulation circuit and converted into a pulse signal, or after the digital signal has been converted through a D/A converter into an analog signal once, it may be input to the pulse modulation circuit.

As an embodiment of the pulse modulation circuit, in the case of analog signal input, an analog input PWM modulation circuit or an analog input ΔΣ circuit can be used; and in the case of a digital signal input, a digital input PWM modulation circuit or a digital input ΔΣ circuit can be used.

Further, the digital amplifier according to the invention may include any of a switching amplifier, a digital amplifier, and a D-class amplifier. Still further, each aspect described above is for embodiments of the invention so the invention does not require each aspect described above, and the invention may use different combinations of aspects.

### BRIEF DESCRIPTION OF THE DRAWINGS

FIG. 1 is a block diagram showing the inner constitution of an audio component in which a digital amplifier of an embodiment of the invention is incorporated;

FIG. 2 is a circuit block diagram showing an inner constitution example of a digital amplifier module shown in FIG. 1;

FIG. 3 is a circuit block diagram showing an inner constitution example of a noise component extraction circuit shown in FIG. 2;

FIG. 4 is a conceptual illustration showing a relation between a low-pass filter and the noise component extraction circuit that are shown in FIG. 3;

FIG. 5 is a circuit block diagram showing an example in which a driver circuit is added to the output of the noise component extraction circuit in FIG. 1;

FIG. 6 is a circuit block diagram showing an example in the case that the noise component extraction circuit in FIG. 5 includes a filter and an amplifier;

FIG. 7 is a circuit block diagram showing an example in the case that an attenuator is added to the noise component extraction circuit in FIG. 6;

FIGS. 8A to 8F are circuit block diagrams showing constitutional examples of a filter shown in FIGS. 6 and 7;

FIG. 9 is a circuit diagram showing a detailed working constitutional example of a digital amplifier according to the invention;

FIG. 10 is a circuit diagram showing a constitutional example in the case that the noise component extraction circuit according to the invention is applied to a switching power supply;

FIG. 11 is a circuit diagram showing an embodiment in the case that noise extraction is performed from the back stage of the low-pass filter;

FIG. 12 is a circuit diagram showing an example in the case that a noise extraction path shown in FIG. 11 is decoupled from a main path;

FIG. 13 is a circuit diagram showing an example in the case that a driver is provided in the noise extraction path shown in FIG. 12;

FIG. 14 is a circuit diagram showing an example in the case that a phase equalizer is provided in the noise component extraction circuit shown in FIG. 11;

FIG. 15 is a circuit diagram showing an example in the case that an attenuator is provided in the noise extraction path shown in FIG. 13; and

FIG. 16 is a circuit diagram showing an example in the case that the noise component extraction circuit shown in FIG. 11 is applied to a switching power supply.

### DETAILED DESCRIPTION OF THE INVENTION

Example embodiments of the invention will be described with reference to attached drawings in detail. The invention is not limited to the below-described embodiments, but the invention covers can be appropriate changes to the embodiments.

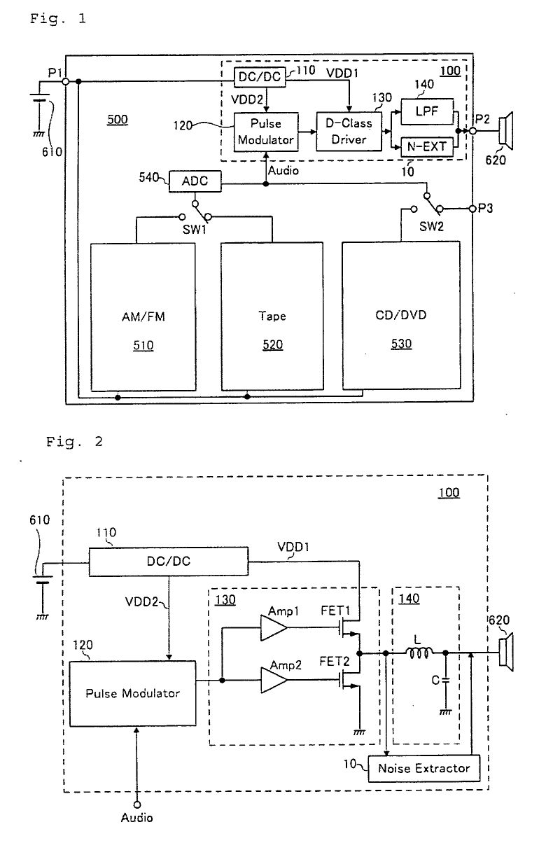

FIG. 1 is a block diagram showing the inner constitution of an audio component in which a digital amplifier of an embodiment of the invention is incorporated. As shown in FIG. 1, the audio component includes an AM/FM radio receiver 510, an audio tape player 520, a CD/DVD player 530, and a digital amplifier module 100 including a digital amplifier circuit 100.

The AM/FM radio receiver 510 and the audio tape player 520, which each outputs an audio signal in an analog format, are connected to a switch SW1, and the signal selected by this switch SW1 is input to an A/D converter 540.

An audio signal in the analog format outputted from the AM/FM radio receiver 510 or the audio tape player 520 is converted into a digital signal by the A/D converter 540, and input to a pulse modulator 120 in the digital amplifier module 100.

The CD/DVD player 530, which outputs an audio signal in a digital format, and a port P3 for inputting a digital signal from the outside are connected to a switch SW2. The signal selected by this switch SW2 is input to the pulse modulator 120 in the digital amplifier module 100.

The digital amplifier module 100 includes a DCDC converter 110, the pulse modulator 120, a D-class driver 130, and a low-pass filter 140. The digital amplifier module 100 amplifies the audio signal inputted from the AM/FM radio receiver 510, the audio tape player 520, the CD/DVD player 530, or the external port P3, and outputs the audio signal in an analog format through a port P2 to a speaker 620 provided for the outside of the audio component 500.

The pulse modulator 120 and the D-class driver 130 are driven by electric power generated by the DCDC converter 110. By use of power supply from a battery 610 provided outside of the audio component 500, the DC/DC converter 110 generates a voltage VDD2 and supplies it to the pulse modulator 120. Further, the DC/DC converter 110 generates a voltage VDD1 and supplies it to the D-class driver 130. The battery 610 is connected to the audio component 500 through a port P1, and supplies the electric power also to the AM/FM radio receiver 510, the audio tape player 520, and the CD/DVD player 530.

For the DC/DC converter 110 and the pulse modulator 120, switching waveform generation circuits are provided respectively. These switching waveform generation circuits generate driving waveforms respectively in accordance with the switching frequency of the DC/DC converter 110 and the switching frequency of the D-class driver 130.

A noise component extraction circuit 10 is connected to the output stage of the D-class driver 130 in parallel with the low-pass filter 140. The noise component extraction circuit 10 extracts a noise component included in the output of the D-class driver 130. The extracted noise component is adjusted in its phase and gain, and thereafter added to the output of the low-pass filter 140. As a result, the noise component that has remained in the audio signal even after the audio signal has passed through the low-pass filter 140 is cancelled.

FIG. 2 is a circuit block diagram showing an example of the inner constitution of the digital amplifier module shown in FIG. 1. As shown in FIG. 2, the D-class driver 130 incorporated in this digital amplifier module 100 includes a high-side switching element FET1, a low-side switching element FET2, and driver amplifiers Amp1 and Amp2 which drive their respective switching elements by a PWM signal from the pulse width modulator 120.

The signal outputted from the D-class driver 130 passes through the low-pass filter 140 composed of an inductor L and a capacitor C, whereby the analog audio signal is extracted, and the speaker 620 is driven by this audio signal.

The noise component extraction circuit 10 extracts the noise component included in the output signal of the D-class driver 130 and adds this extracted noise component to the output of the low-pass filter 140.

Namely, the output pulse of the D-class driver 130 flows in a main path through the low-pass filter 140 and in a noise path trough the noise component extraction circuit 10. The signals processed respectively in the main path and the noise path are synthesized and thereafter inputted to the speaker 620.

Thus, by synthesizing the audio signal obtained through the low-pass filter 140, and the noise signal obtained through the noise component extraction circuit 10, the noise component included in the audio signal can be removed.

FIG. 3 is a circuit block diagram showing an inner constitutional example of the noise component extraction circuit shown in FIG. 2. As shown in FIG. 3, the noise component extraction circuit 10 includes a band limiting section 12 that limits a frequency band that is an extraction target, a phase adjustment section 14 which adjusts the phase of the extracted noise component, and a gain adjustment section 16 which adjusts the gain of the extracted noise component.

The band limiting part 12 extracts a frequency component of a switching carrier, and the phase adjustment part 14 inverts, in relation to a ripple noise component included in the output of the low-pass filter 140, the phase of the extracted carrier component in a position of a calculation part 18 which constitutes an addition point.

The gain adjustment part 16 matches the amplitude level of the extracted carrier component with the amplitude level of the ripple noise component included in the output of the low-pass filter 140 in the position of the calculation part 18 which constitutes the addition point. Namely, in the noise component extraction circuit 10, a reverse-phase signal of the noise component included in the output of the D-class driver 130 is generated, and added to the output of the low-pass filter 140, whereby the noise included in the audio signal is canceled.

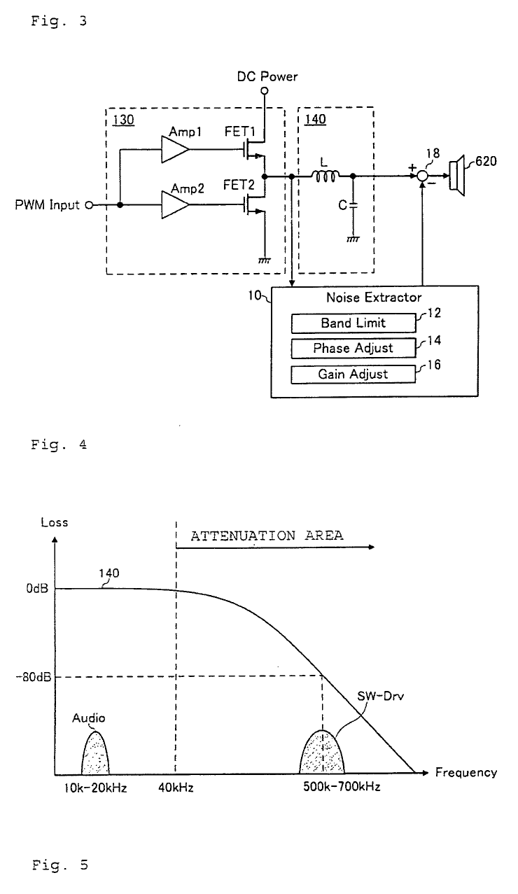

FIG. 4 is a characteristic diagram showing a relationship between the low-pass filter and the noise component extraction circuit that are shown in FIG. 3. In this characteristic diagram, insertion loss is plotted on the vertical axis, and the frequency is plotted on the horizontal axis. As shown by hatched bands in FIG. 4 in the signals used in the digital amplifier circuit, there are an audio signal Audio having a band of 10 kHz to 20 kHz, and a switching signal SW-Drv of a D-class driver having a band of 500 kHz to 700 kHz.

Of these signals, a ripple noise removal target is the switching signal SW-Drv of the D-class driver. This switching signal is set in an attenuation area of the low-pass filter 140. In the example shown in FIG. 4, the cutoff frequency of the low-pass filter 140 is set to 40 kHz, and an area of the frequency higher than 40 kHz is taken as the attenuation area.

Here, in even the signals set in the same attenuation area, since the low-pass filter 140 has an attenuation characteristic having a frequency inclination according to the second degree, the noise components that are different in frequency area are different in attenuation amount. For example, since the switching signal SW-Drv of the D-class driver varies in the band with a width, after it has passed through the low-pass filter 140, the noise level in even the same carrier varies according to variation of the frequency. Accordingly, in order to obtain the cancellation level of each ripple noise exactly, it is effective to give a level adjustment function corresponding to the attenuation characteristic of the low-pass filter 140 to the noise component extraction circuit.

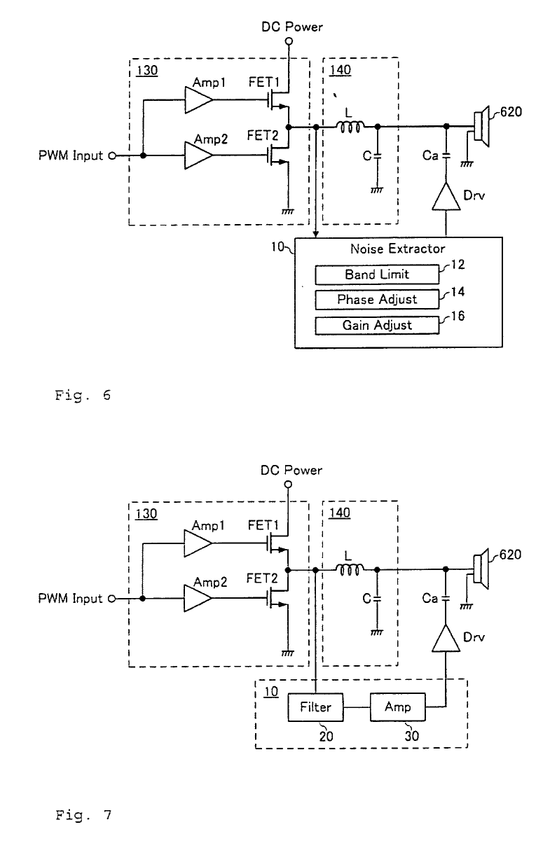

FIG. 5 is a circuit block diagram showing an example in which a driver circuit is added to the output of the noise component extraction circuit in FIG. 1. The main path including the low-pass filter 140 is generally small in impedance. Therefore, it is preferable, as shown in FIG. 5, to provide a driver circuit Drv in the back stage of the noise component extraction circuit 10, and add the extracted noise component to the main path by capacitative coupling Ca. FIG. 6 is a block diagram showing an example in the case that the noise component extraction circuit in FIG. 5 includes a filter and an amplifier. As shown in FIG. 6, it is preferable that the noise component extraction circuit 10 consists of a filter 20 and an amplifier 30 which can appropriately perform band limiting, phase adjustment, and gain adjustment for the noise component that is a cancellation target.

FIG. 7 is a circuit block diagram showing an example in the case that an attenuator is added to the noise component extraction circuit in FIG. 6. In the case that the pulse output level of the D-class driver 130 is large, it is preferable as shown in FIG. 7 to provide an attenuator 50 in the front stage of the noise component extraction circuit 10 to control the input level.

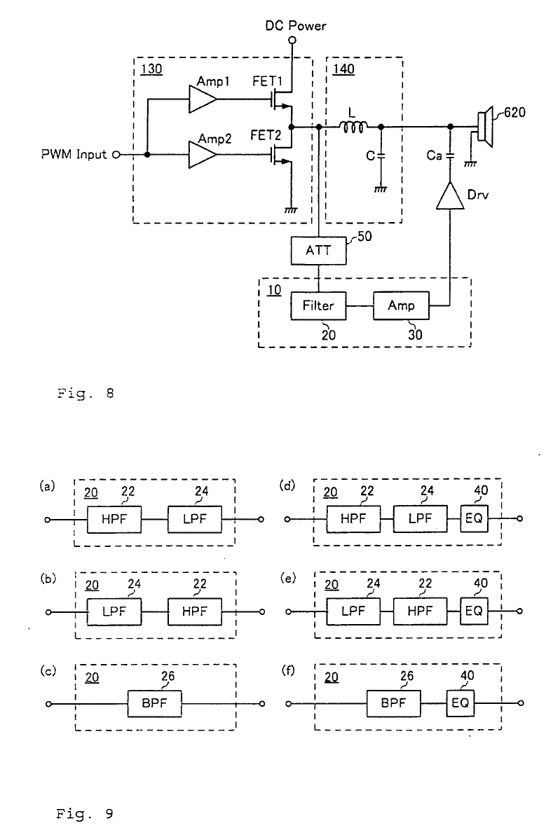

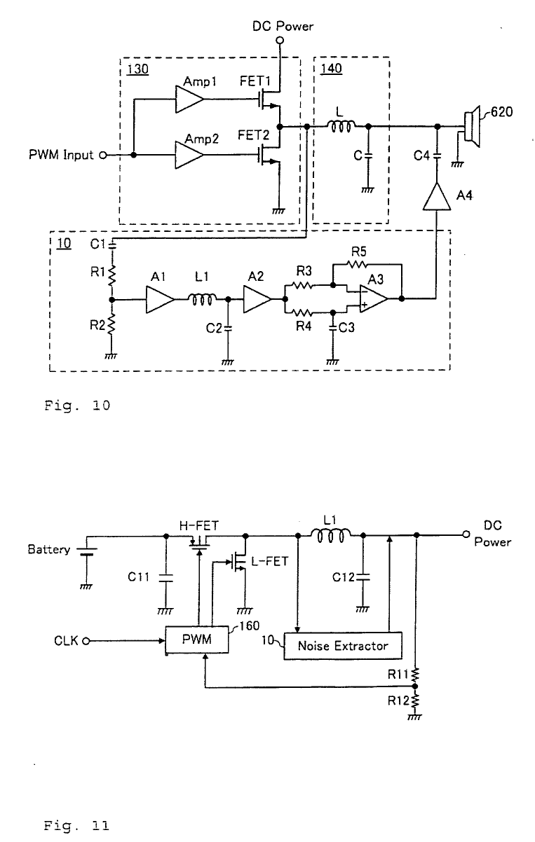

FIGS. 8A to 8F are circuit block diagrams showing constitutional examples of the filter 20 shown in FIGS. 6 and 7. FIGS. 8A and 8B show examples in which the band limiting and the phase adjustment are performed in combination with a high-pass filter 22 and a low-pass filter 24. FIG. 8C shows an example in which the filter 20 is constituted by means of a band-pass filter 26. FIGS. 8D to 8F are examples in which, in order to facilitate the phase adjustment, phase equalizers are added to the circuits shown in FIGS. 8A to 8C respectively.

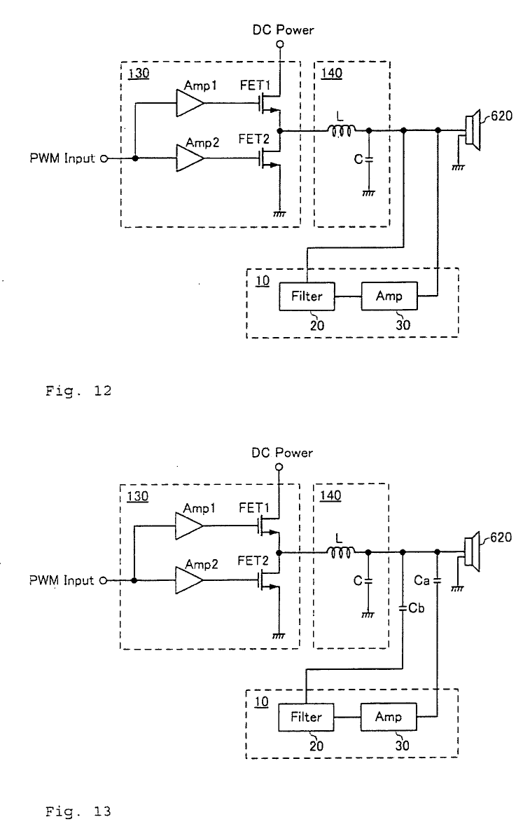

FIG. 9 is a circuit diagram showing a detailed working constitutional example of the digital amplifier according to the invention. In the example noise component extraction circuit 10 shown in FIG. 9, a capacitor C1 and resistors R1, R2 constitute an attenuator and a high-pass filter; an inductor L1 and a capacitor C2 constitute a low-pass filter, and an amplifier A3, resistors R3 to R5, and a capacitor C3 constitute a phase equalizer. Amplifiers A1 and A2 are buffer amplifiers.

For the low-pass filter composed of the inductor L1 and the capacitor C2, the low-pass filter is provided with the same attenuation characteristic as that of the low-pass filter 140 provided in the main path.

An amplifier A4 provided in the output stage of the noise component extraction circuit 10 constitutes a driver, and a capacitor C4 provided in the back stage of the amplifier A4 constitutes an adder thereby to add the extracted noise component to the main path.

FIG. 10 is a circuit diagram showing a constitutional example in the case that the noise component extraction circuit according to an embodiment of the invention is applied to a switching power supply. As shown in FIG. 10, this switching power supply includes a capacitor C11 connected to a battery, a high-side switch H-FET, a low-side switch L-FET, a PWM circuit 160 which controls these switches, a coil L1 and a capacitor C12 that smooths the outputs of the switches, and resistors R11 and R12 which divides an output voltage and feeds back the divided output voltage to the PWM circuit 160.

In the PWM circuit 160, a clock signal CLK is input from the outside the circuit 160. The PWM circuit 160, while monitoring an output voltage, generates respective drive waveforms of the switching elements H-FET and L-FET by means of the inputted clock signal CLK. Further, it is preferable that each switch shown in FIG. 10 is composed of an FET.

In the case that the noise component extraction circuit 10 according to an embodiment of the invention is applied to the constructed switching power supply, it is advantageous that the signal taken out from the front stage of the inductor L1 and the capacitor C12, which smooths the outputs of the switches, is input to the noise component extraction circuit 10, and the extracted noise component is added to the output of the inductor L1 and the capacitor C12.

FIG. 10. illustrates an example embodiment of the case where the invention is applied to a step-down switching power supply. However, the invention can be applied also to other switching circuits such as a step-up switching power supply, and a step-up/step-down switching power supply.

FIG. 11 is a circuit diagram showing an embodiment in the case that the noise extraction is performed from the back stage of the low-pass filter 140. As shown in FIG. 11, the noise component extraction circuit 10 may be provided in the back stage of the low-pass filter 140. In this case, the noise is extracted from the back stage of the low-pass filter 140, and this extracted noise is added to the front stage of the speaker 620.

The noise component extraction circuit 10 includes a filter 20 and an amplifier 30. The filter 20 extracts an unnecessary noise component that is a cancellation target from an output signal of the low-pass filter 140. The gain of the extracted noise component is adjusted by the amplifier circuit 30, and the adjusted noise component is added to the original output line in the front stage of the speaker 620.

Here, by using an inverting amplifier for the amplifier 30, the phase of the extracted noise component is inverted, and the noise component is subtracted from the main audio signal at an output connection part of the noise component extraction circuit 10, so that the unnecessary noise component included in the audio signal such as the switching component is reduced.

The embodiment shown in FIG. 11 in which the noise component is extracted from the back stage of the low-pass filter, compared with the previous embodiment in which the noise component is extracted from the front stage of the low-pass filter, provides easy phase adjustment of an antiphase cancellation signal to be added to the main audio signal and level adjustment thereof. This phase adjustment is performed by the filter 20 in FIG. 11, and the level adjustment is performed by the amplifier 30 in FIG. 11. Further, the filter 20 may be composed of a band-pass filter that selects a noise band to be extracted.

FIG. 12 is a circuit diagram showing an example in the case that the noise extraction path shown in FIG. 11 is decoupled from the main path. As shown in FIG. 12, the circuit may be implemented by providing decoupling capacitors Ca and Cb for the connection part of the noise component extraction circuit 10 so that large current is not permitted to flow in the noise extraction circuit to reduce an influence on the main signal.

FIG. 13 is a circuit diagram showing an example in the case that a driver is provided in the noise extraction path shown in FIG. 12. As shown in FIG. 13, the driver Drv may be provided in the back stage of the noise component extraction circuit 10, and the output of the noise component extraction circuit 10 is added through the capacitative coupling Ca to the output line. This constitution is effective for the case where the impedance of the output line is small.

FIG. 14 is a circuit diagram showing an example in the case that a phase equalizer is provided in the noise component extraction circuit shown in FIG. 11. As shown in FIG. 14, the constitution in which the phase difference when the subtraction is performed in the output line is matched by a phase equalizer 40 may be adopted.

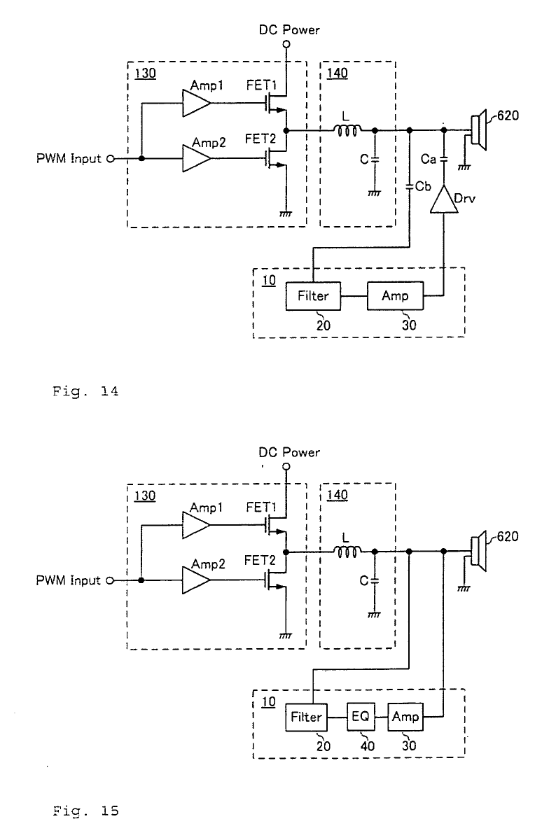

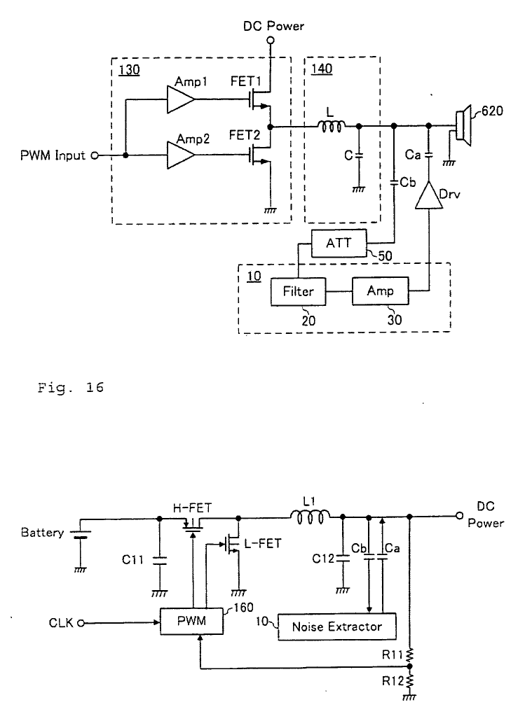

FIG. 15 is a circuit diagram showing an example in the case that an attenuator is provided in the noise extraction path shown in FIG. 13. As shown in FIG. 15, in the case that the input level is large, an attenuator 50 may be provided in the front stage of the noise component extraction circuit 10.

The filter 20 shown in FIGS. 11 to 15 can have various constructions, including modifications to the filters shown in FIG. 8.

FIG. 16 is a circuit diagram showing an example in the case that the noise component extraction circuit shown in FIG. 11 is applied to a switching power supply. As shown in FIG. 16, in the case that the noise component extraction circuit is applied to the switching power supply, the noise component extraction circuit 10 is connected to the back stage of a smooth filter composed of an inductor L1 and a capacitor C12, and the extracted reverse-phase noise signal is added to the output line of the switching power supply circuit. Also in this constitution, it is preferable to connect the noise component extraction circuit 10 through decoupling capacitors Ca and Cb to the main output line.

As described above, according to embodiments of the invention, since a noise component for cancellation can be extracted/reproduced with high accuracy, it is possible to achieve a noise level necessary for a digital amplifier and a switching power supply that are used for the purpose of high output and high grade.

According to embodiments of the invention, the noise component for cancellation can be extracted and reproduced with high accuracy. Therefore, the invention may be applied to a high-grade digital amplifier, switching power supply, or other devices.

What is claimed is:

1. A noise reduction circuit comprising:
a switch coupled to an input signal;
a filter coupled to the back stage of the switch; and
a noise component extraction circuit adapted to extract a noise component included in either an output of the switch or an output of the filter to generate a reverse-phase noise component, and add the reverse-phase noise component to the output of the filter.

2. The noise reduction circuit according to claim 1, wherein the noise component extraction circuit is decoupled from the output of the switch.

3. The noise reduction circuit according to claim 1, wherein the noise component extraction circuit is decoupled from the output of the filter.

4. A digital amplifier which switch-amplifies an audio signal and performs analog output, the digital amplifier comprising:
a pulse modulation circuit adapted to modulate the audio signal;
a D-class drive circuit driven by the output of the modulation circuit and generates a drive output;
a filter adapted to smooth the drive output of the D-class drive circuit; and
a noise component extraction circuit adapted to extract a noise component from the output of the D-class drive circuit and add the extracted noise component to the output of the filter, wherein
the noise component extraction circuit includes an attenuator, the attenuator adapted to attenuate the extracted noise component correspondingly to at least an attenuation characteristic of the filter.

5. The digital amplifier according to claim 1, wherein the output of the noise component extraction circuit is added through a DC cut capacitor to the output of the filter.

6. The digital amplifier according to claim 1, wherein the output of the filter has a low impedance and the noise component extraction circuit includes a phase compensation circuit adapted to compensate a phase error produced by addition of the extracted noise component to low impedance output of the filter.

7. The digital amplifier according to claim 1, wherein the noise component extraction circuit includes:
a band limiting section to extract the noise component;
a phase compensation section to compensate for a phase of the extracted noise component; and
a gain adjustment section to adjust a gain of the extracted noise component correspondingly to the attenuation characteristic of the filter.

8. The digital amplifier according to claim 1, wherein the noise component extraction circuit includes:
a high-pass filter adapted to extract the noise component; and
a low-pass filter having the same attenuation characteristic as the attenuation characteristic of the filter.

9. The digital amplifier according to claim 4, wherein the noise component extraction circuit includes:
an amplifier adapted to amplify an output level of the low-pass filter; and
a DC cut capacitor adapted to block a DC component from the output of the low-pass filter.

10. A switching power supply which controls a switch arranged between a power source and a load thereby to perform power conversion, the power supply comprising:
a control circuit adapted to control the switch;
a filter adapted to smooth the output of the switch; and
a noise component extraction circuit adapted to extract a noise component from the output of the switch or an output of the filter to generate a reverse-phase noise component, and add the reverse-phase noise component to the output of the filter.
